SEQUENCE LISTING (2 sequences)

SEQ ID NO: 1 - DNA, 60 bp, artificial sequence
  multiple cloning site region
gccaagcttgcatgcctgcaggtcgactctagaggatccccgggtaccgagctcgaattc

SEQ ID NO: 2 - DNA, 3480 bp, Artificial Sequence
  2kb yeast rDNA probe
tgccgcttcactcgccgttactaaggcaatcccggttggtttcttttcctccgcttattgatatgcttaagttcagcgggtactcctacctgatttgaggtcaaactttaagaacattgttcgcctagacgctctcttcttatcgataacgttccaatacgctcagtataaaaaaagattagccgcagttggtaaaacctaaaacgaccgtacttgcattatacctcaagcacgcagagaaacctctctttggaaaaaaaacatccaatgaaaaggccagcaatttcaagttaactccaaagagtatcactcactaccaaacagaatgtttgagaaggaaatgacgctcaaacaggcatgccccctggaataccaaggggcgcaatgtgcgttcaaagattcgatgattcacggaattctgcaattcacattacgtatcgcatttcgctgcgttcttcatcgatgcgagaaccaagagatccgttgttgaaagtttttaatattttaaaatttccagttacgaaaattcttgtttttgacaaaaatttaatgaatagataaaattgtttgtgtttgttacctctgggccccgattgctcgaatgcccaaagaaaaagttgcaaagatatgaaaactccacagtgtgttgtattgaaacggttttaattgtcctataacaaaagcacagaaatctctcaccgtttggaatagcaagaaagaaacttacaagcctagcaagaccgcgcacttaagcgcaggcccggctggactctccatctcttgtcttcttgcccagtaaaagctctcatgctcttgccaaaacaaaaaaatccattttcaaaattattaaatttctttaatgatccttccgcaggttcacctacggaaaccttgttacgacttttagttcctctaaatgaccaagtttgtccaaattctccgctctgagatggagttgcccccttctctaagcagatcctgaggcctcactaagccattcaatcggtactagcgacgggcggtgtgtacaaagggcagggacgtaatcaacgcaagctgatgacttgcgcttactaggaattcctcgttgaagagcaataattacaatgctctatccccagcacgacggagtttcacaagattaccaagacctctcggccaaggttagactcgctggctccgtcagtgtagcgcgcgtgcggcccagaacgtctaagggcatcacagacctgttattgcctcaaacttccatcggcttgaaaccgatagtccctctaagaagtggataaccagcaaatgctagcaccactatttagtaggttaaggtctcgttcgttatcgcaattaagcagacaaatcactccaccaactaagaacggccatgcaccaccacccacaaaatcaagaaagagctctcaatctgtcaatccttattgtgtctggacctggtgagtttccccgtgttgagtcaaattaagccgcaggctccactcctggtggtgcccttccgtcaattcctttaagtttcagccttgcgaccatactccccccagaacccaaagactttgatttctcgtaaggtgccgagtgggtcattaaaaaaacaccacccgatccctagtcggcatagtttatggttaagactacgacggtatctgatcatcttcgatcccctaactttcgttcttgattaatgaaaacgtccttggcaaatgctttcgcagtagttagtcttcaataaatccaagaatttcacctctgacaattgaatactgatgcccccgaccgtccctattaatcattacgatggtcctagaaaccaacaaaatagaaccaaacgtcctattctattattccatgctaatatattcgagcaatacgcctgctttgaacactctaattttttcaaagtaaaagtcctggttcgccaagagccacaaggactcaaggttagccagaaggaaaggccccgttggaaatccagtacacgaaaaaatcggaccggccaaccgggcccaaagttcaactacgagctttttaactgcaacaactttaatatacgctattggagctggaattaccgcggctgctggcaccagacttgccctccaattgttcctcgttaaggtatttacattgtactcattccaattacaagacccgaatgggccctgtatcgttatttattgtcactacctccctgaattaggattgggtaatttgcgcgcctgctgccttccttggatgtggtagccgtttctcaggctccctctccggaatcgaacccttattccccgttacccgttgaaaccatggtaggccactatcctaccatcgaaagttgatagggcagaaatttgaatgaaccatcgccagcacaaggccatgcgattcgaaaagttattatgaatcatcaaagagtccgaagacattgattttttatctaataaatacatctcttccaaagggtcgagattttaagcatgtattagctctagaattaccacagttataccatgtagtaaaggaactatcaaataaacgataactgatttaatgagccattcgcagtttcactgtataaattgcttatacttagacatgcatggcttaatctttgagacaagcatatgactactggcaggatcaaccagataactatcttaaaagaagaagcaacaagcagtaaaaaagaaagaaaccgaaatctctttttttttttcccacctattccctcttgctagaagatacttattgagtttggaaacagctgaaattccagaaaaattgctttttcaggtctctctgctgccggaaatgctctctgttcaaaaagcttttacactcttgaccagcgcactccgtcaccataccatagcactctttgagtttcctctaatcaggttccaccaaacagataccccggtgtttcacggaatggtacgtttgatatcgctgatttgagaggaggttacacttgaagaatcacagtcttgcgaccggctattcaacaaggcattcccccaagtttgaattctttgaaatagattgctattagctagtaatccaccaaatccttcgctgctcaccaatggaatcgcaagatgcccacgatgagactgttcaggttaaacgcaaaagaaacacactctgggaatttcttcccaaattgtatctctcaatacgcatcaacccatgtcaattaaacacgctgtatagagactaggcagatctgacgatcacctagcgactctctccaccgtttgacgaggccatttacaaaaacataacgaacgacaagcctactcgaattcgtttccaaactcttttcgaacttgtcttcaactgctttcgcatgaagtacctcccaactacttttcctcacacttgtactccatgactaaaccccccctcccattagccaagcttgcatgcctgcaggtcgactctagaggatccccgggtaccgagctcgaattc

(19) United States
(12) Patent Application Publication
(10) Pub. No.: US 2003/0134311 A1
(43) Pub. Date: Jul. 17, 2003

(54) Nucleic acid-based method for tree phennotype prediction

(76) Inventors: Simon Potter, Coquitlam (CA); Paul A. Watson, Vancouver (CA)

Correspondence Address: OGILVY RENAULT, 1981 MCGILL COLLEGE AVENUE, SUITE 1600, MONTREAL, QC, H3A2Y3, CA

(21) Appl. No.: 10/297,621

(22) PCT Filed: Jun. 21, 2001
(86) PCT No.: PCT/CA01/00927

(30) Foreign Application Priority Data
Jun. 23, 2000 (US) 60213585

(51) Int. Cl. C12Q 1/68; A01H 1/00

(52) U.S. Cl. 435/6; 800/284

(57) ABSTRACT

The present invention relates to a novel method for the prediction of fibre length and the rapid selection of superior trees for given pulp and paper product lines using a DNA probe. The method comprises the isolation of tree genomic DNA from a hybrid spruce live tissue source, hybridization of the spruce DNA probe to that genomic DNA and the densitometric assessment of the intensity of the hybridization pattern obtained. This determines the precise degree of genetic admixing (or introgression) of the two parent species within the hybrid population. Due to the linear relationship—in the hybrid spruce population examined—between degree of genetic introgression and fibre length (discovered in this method), the intensity of the DNA probe hybridization pattern can be used to directly, accurately and reproducibly predict the fibre length found (for a given tree age) within an individual hybrid spruce within the population.

### BACKGROUND OF THE INVENTION

(a) Field of the Invention

This invention is in the fields of molecular biology and tree improvement, and pulp and paper properties evaluations. This invention allows for an enhanced efficiency of selection for trees with given fibre properties from both natural and plantation populations for specific pulp and paper product lines.

(b) Description of Prior Art

It has long been recognized that introgression (a natural form of genetic hybridization) commonly occurs between three spruce species—Sitka (Picea sitchensis), White (P. glauca) and Engelmann (P. engelmannii) (Daubenmire, R. (1968) Can. J. Bot. 46:787-798). The areas in which this hybridization occurs are known as “introgression zones”, an example of which can be found in and around the Nass Skeena Transition of British Columbia (FIG. 1). Two main groups of such natural hybrids occur in this zone: “interior” hybrids (which are predominantly mixtures of White and Engelmann spruce) and interior X Sitka hybrids (Sutton, B. C. S., et al., (1991) Theor. Appl. Genet. 82:242-248), although the exact extent of the zone is not clear and the representation of genotypes (parentage) within it is poorly defined (Roche, L. (1969) New Phytol. 68:505-554).

Given the fact that interior and Sitka spruce are reported to have substantially different basic fibre properties—interior: fine fibred, 2.0-3.5 mm×25-30 μm; Sitka: coarse fibred, 3.2-5.6 mm×35-45 μm (Isenberg, I. H. Pulp woods of the United States and Canada, Vol. I—Softwoods. 3^{rd} ed. Inst. Pap. Chem. Appleton, Wis. (1981))—it is reasonable to suppose that the hybrid trees found in introgression zones may inherit these properties to varying degrees dependent on the degree of hybridization between the species.

In order to characterize these zones and to test whether the natural genetic hybridization has any effects on the wood quality of these trees, it is a critical pre-requisite that methods be developed for determining the degree of hybridization in the zones on a tree-by-tree basis. Usually, tree identification is done on the basis of visual examinations of gross morphological characters. However, this technique can prove inaccurate when attempting to distinguish between closely related but genetically distinct hybrids. Methods such as isozyme analysis may be of some use but can be time consuming and laborious and may lack the precision required to distinguish closely related clones (Murphy, R. W. Proteins I: Isozyme electrophoresis. In: Hillis, D. M.; Moritz, C., eds. Molecular Systematics. Sunderland, Mass., U.S.A.: Sinauer Associates, 45-126 (1990)). Better by far for distinguishing hybrid trees from one another are methods which discriminate on the basis of DNA polymorphisms (tiny changes in the structure of the genes each tree possesses) (Potter, S. (1998) PPR 1356).

Previous studies have reported the isolation of species-specific DNA probes for interior and Sitka spruce based on organellar (chloroplast and mitochondrion) inheritance patterns (Sutton, B. C. S. et al., (1993) Can. J. For. Res. 24:278-285) which can be used to fingerprint clones on an interior/Sitka basis. These probes, however, can give no information on degrees of hybridization found in introgressed clones. Probes based on nuclear sequences have also been used with varying success—for example, ribosomal DNA (rDNA) regions of red and black spruce are known to exhibit intraspecies variation of some polymorphic regions which have been exploited to produce species-specific RAPD markers. By their nature, these markers are based on arbitrary sequences whose general applicability (i.e. across species) is difficult to determine (Bobola, M. S. et al.,(1992) Mol. Biol. Evol. 9:125-137), Perron, M. et al., (1997) Molecular ecology 6:725-734).

In contrast to the situation reported for red and black spruce, the present invention describes a nucleic acid probe developed for interior/Sitka hybrids (in the Nass-Skeena introgression zone) which is capable of quantitatively estimating the degree of genetic hybridization between the two. This probe, Eco2.0, is based on a stretch of yeast (Saccharomyces cerevisiae) 18S rDNA and detects species-specific 18S external transcribed spacer (ETS) polymorphisms in a quantitative fashion as determined by densitometric image analysis.

It would be highly desirable to be provided with a new method for identification of tree lineages having superior phenotype.

### SUMMARY OF THE INVENTION

One aim of the present invention is to provide a method for identifying individual trees having superior phenotype.

In accordance with the present invention there is provided a Eco2.0 probe, its purification and sequence characterization, and and its use as a marker for the degree of introgression in individual hybrids within the Nass-Skeena zone.

a) the Eco2.0 probe has been isolated and cloned into E. coli

b) the probe has been purified and partially sequenced to determine it's identity.

c) the utility of the probe as a marker for the fibre quality of the hybrids has been assessed by a direct comparison of Southern blot band intensity patterns to fibre property parameters for individual tree samples selected from geographical areas spanning the Nass-Skeena introgression zone.

Still in accordance with-the present invention, there is provided a method for identifying tree lineage capable of expressing desired biological and/or biochemical phenotypes comprising the steps of:

a) obtaining a nucleic acid sample, such as RNA or DNA, from trees of pure species and/or hybrid thereof;

b) obtaining a restriction pattern of restriction fragments by subjecting said nucleic acid sample of step a) to at least one restriction enzyme, wherein said restriction enzyme maximizes differences between said restriction pattern of pure species and/or hybrid thereof;

c) visualizing said restriction pattern of step b) by submitting the treated nucleic acid sample of step b) to at least one labeled probe, such as without limitation Eco2.0, for complementary hybridization between said probe and said nucleic acid sample, wherein said probe allows for detection of the degree of hybridization and/or different intensity of said restriction fragments between trees of pure species and/or hybrid thereof; and

d) correlating said restriction pattern and/or intensity of restriction fragments of step c) to at least one selected biological and biochemical phenotype of said tree, wherein said phenotype is associated with a genetic locus correlated with said phenotype.

Step d) may further comprise a standard curve for predictive relationship between said restriction pattern and/or intensity of restriction fragments, and said phenotype.

The tree of pure species and/or hybrid thereof may be naturally or artificially produced.

The nucleic acid sample of step a) may be obtained from a leaf, cambium, root, bud, stem, cork, phloem or xylem.

The tree may be of the genus Picea, or may be of a genus selected from the group consisting of Populus, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja, and Canya.

Step c) may further comprise measurement of intensity of said restriction fragments.

The biological or biochemical phenotype searched for may be for example selected from the group consisting of fiber length, wood density, fiber collapsibility, fiber coarseness, cell wall thickness, growth rate, lignin content, guaiacyl lignin content, syringyl lignin content, carbohydrate content, kraft pulp yield, mechanical pulp energy demand, chemical uptake for chemical pulping, extractive content, and extractive compounds.

Also in accordance with the present invention, there is provided the use of a pattern of restriction fragments obtained by subjecting a nucleic acid sample from trees of pure species and/or hybrid thereof to at least one restriction enzyme for identifying tree lineage capable of expressing desired biological and/or biochemical phenotypes, wherein said restriction enzyme maximizes differences between said pattern of restriction fragments from pure species and/or hybrid thereof.

Still in accordance with the present invention, there is provided a pattern of restriction fragments obtained by subjecting a nucleic acid sample from trees of pure species and/or hybrid thereof to at least one restriction enzyme for use in identifying tree lineage capable of expressing desired biological and/or biochemical phenotypes, wherein said restriction enzyme maximizes differences between said pattern of restriction fragments from pure species and/or hybrid thereof.

Further in accordance with the present invention, there is provided a method of screening a plurality of trees of diverse phenotypes which comprises the steps of:

a) Characterizing wood quality of at least two trees with different degree of hybridization;

b) Developing a standard curve from said trees for a predictive relationship between restriction patterns and phenotypes;

c) assessing a plurality of natural species, hybrids for restriction patterns of a plurality of hybridization markers;

d) comparing said restriction patterns of said hybrids with said standard curve to deduce a phenotype of said hybrids; and

e) harvesting said hybrids based on predicted phenotypes.

In accordance with the present invention, there is also provided a method of producing a plurality of clonal trees having predictable, consistent and/or enhanced wood or fibre quality properties, which comprises the steps of:

a) characterizing wood quality of at least two trees with different degree of hybridization;

b) developing a standard curve from said trees for a predictive relationship between restriction patterns and phenotypes;

c) assessing a plurality of natural species hybrids for restriction patterns of a plurality of hybridization markers;

d) comparing said restriction patterns of said hybrids with said standard curve to deduce a phenotype of said hybrids;

e) obtaining a plurality of progeny trees from said parental trees by performing cross-pollination or somatic embryogenesis; and

propagating somatic embryos of said progeny trees obtained in step e) to produce a plurality of clonal trees, essentially all of said clonal trees having predictable, consistent and/or enhanced wood or fibre quality properties.

The present invention further provides a stand of clonal trees with enhanced wood or fibre properties produced by the method of the present invention, the genome of said trees containing a restriction pattern, said restriction pattern being the same restriction pattern associated with said enhanced wood or fibre properties.

In accordance with the present invention, the Eco2.0 probe may also be used as a marker for predicting fiber quality of tree samples of pure species and/or hybrid thereof.

For the purpose of the present invention the following terms are defined below.

The term “locus” is intended to mean the position occupied on the chromosome by the gene representing a particular trait. The various alternate forms of the gene—that is, the alleles used in mapping—all reside at the same location.

The term “restriction fragment length polymorphism (RFLP)” as used herein means a restriction map that identifies a linear series of sites in DNA, separated from one another by actual distance along the nucleic acid sequence. A restriction map can be obtained for any sequence of DNA, irrespective of whether mutations have been identified in it, or, indeed, whether we have any knowledge of its function. A difference in restriction maps between two individuals can be used as a genetic marker in exactly the same way as any other marker. To relate the restriction map to the genetic map, we must compare the restriction maps of wild-type and corresponding variant, or phenotypes.

The term “restriction fragments” as used herein means fragments of nucleic acid generated following digestion of a purified nucleic acid with restriction enzymes. Restriction fragments may be considered as restriction markers, that are not restricted to those changes that affect a phenotype, they provide the basis for an extremely powerful technique for identifying genetic loci at the molecular level.

The term “hybridization”, or renaturation, as used herein means the ability of two separated complementary strands to reform into a double helix. Renaturation depends on specific base pairing between the complementary strands. The reaction takes place in two stages. First, DNA single strands in the solution encounter one another by chance; if their sequences are complementary, the two strands base pair to generate a short double-helical region. Then the region of base pairing extends along the molecule be a zipper-like effect to form a lengthy duplex molecule. Renaturation describes the reaction between two complementary sequences that were separated by denaturation. However, the technique can be extended to allow any two complementary nucleic acid sequences to anneal with each other to form a duplex structure. The reaction is generally described as hybridization when nucleic acids from different sources are involved, as in the case when one preparation consists of DNA and one of RNA. The ability of two nucleic acids preparations to hybridize constitutes a precise test for their sequence complementarity.

The term “predictive relationship” as used herein means a correlating factor based on a standard curve established between i) a restriction pattern and/or intensity of restriction fragments, and a particular phenotype; or ii) a “random amplified polymorphic DNA” (RAPD) pattern and a particular phenotype.

The term “hybrid thereof” as used herein means a progeny issued from the interbreeding of trees of different breeds, varieties, species, especially as produced through tree crossbreeding for specific genetic and phenotypic characteristics. A hybrid thereof is derived by crossbreeding two different tree species.

### BRIEF DESCRIPTION OF THE DRAWINGS

FIG. 1 illustrates a map of British Columbia spruce species distribution showing the Nass Skeena Transition introgression zone;

FIG. 2 illustrates a map of sampling sites across the Introgression zone. Black and grey circles show sampling sites taken for previous study. Red circles indicate sites sampled for the present study;

FIG. 3A illustrates the pEco2.0 plasmid map. Multiple cloning site region is exploded to show the precise ordering of restriction sites. Insertion point for the 2 kb yeast rDNA probe is indicated;

FIG. 3B illustrates an agarose gel analysis of the plasmid and probe insert. Lanes are described in the text. C: Agarose gel analysis of PCR products using M13 F/R primers. Lanes L to R: markers, PCR products using different proprietary buffers;

FIG. 4 illustrates the sequence of the 2 kb yeast rDNA probe. Bold sequence represents the probe, ends of which were determined and found to be identical to Genbank Z73326;

FIG. 5 illustrates the sequence analysis of the Eco2.0 plasmid using M13 r and f primers;

FIG. 6 illustrates: Panel B—Southern banding pattern and densitometric analysis of the hybridization of the probe to total interior and Sitka spruce hybrid DNA digests. Lanes are described in the text. * Sample from site 2 underloaded. In the text, the bands seen are referred to as Bands 1-5 from the top down. Panels A and C show typical banding patterns for the pure species and densitometry of those patterns;

FIG. 7 illustrates fibre properties as a function of tree age. The 60-80 age class was chosen for comparative purposes as this is the region free from juvenile wood for all samples;

FIG. 8 illustrates fibre coarseness versus length-weighted fibre length showing a strong positive correlation;

FIG. 9 illustrates a scatterplot matrix for fibre properties, site index and band intensities data.

FIG. 10 illustrates the relationship between Band intensity and length-weighted fibre length (LWFL) in the spruce hybrids and indicates an excellent linear relationship between Band 2 and LWFL;

FIG. 11 illustrates a standard curve for relationship between relative Band 2 intensity and length-weighted fibre length obtained from Blot 1 (FIG. 5). Dotted lines show the Band 2 intensity values for spruce samples probed on Blot 2 used in the predictive analysis for fibre length;

FIG. 12 illustrates the second hybridization analysis (Blot 2) used for fibre length prediction experiment. Band 2 is indicated. Lane 1, pEco2.0 plasmid insert positive control; lane 2, Nass-Skeena “Black spruce” sample; lanes 3-8, trees 7-9, 6-4, 5-8, 4-5, 2-4; and

FIG. 13 illustrates that fibre length as a function of site productivity shows no global correlation. Specific biogeoclimatic zones are delineated to emphasize possible intra-zonal correlations.

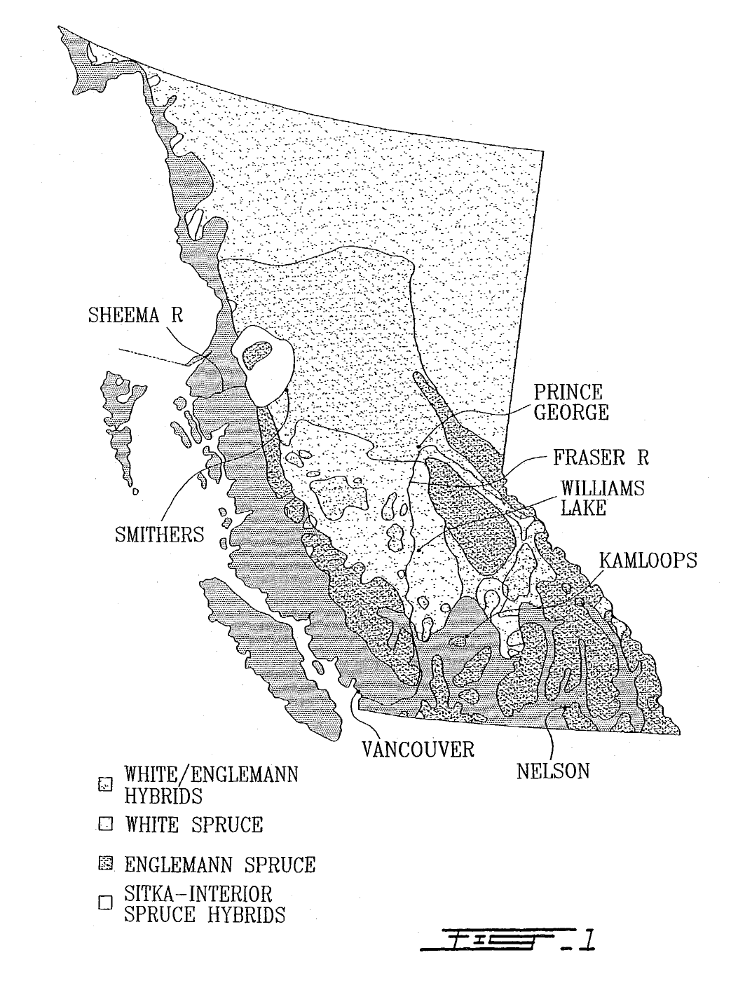

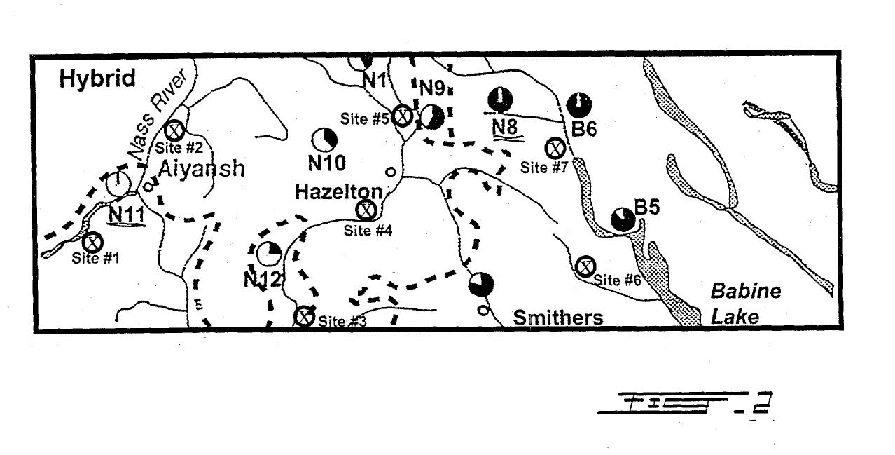

### DETAILED DESCRIPTION OF THE INVENTION

In accordance with the present invention, there is provided a novel method for the prediction of selected phenotypes and the rapid selection of superior trees for given pulp and paper product lines using a DNA probe. The method comprises the isolation of tree genomic DNA from a spruce live tissue source, hybridization of the DNA probe to that genomic DNA and the densitometric assessment of the intensity of the hybridization pattern obtained.

A particular embodiment of the invention is the determination of the precise degree of genetic admixing (or introgression) of the two parent species within the hybrid population. Due to the linear relationship—in the hybrid spruce population for example—between degree of genetic introgression and fibre length (with regard to the present invention), the intensity of the DNA probe hybridization pattern is used to directly, accurately and reproducibly predict the fibre length found (for a given tree age) within an individual hybrid Sitka/Interior spruce within the population.

A preferred embodiment of the invention is the use of DNA probes as predictive tools in forestry tree improvement programs as it represents the first successful practical demonstration of such an application.

One embodiment of the present invention is to provide a novel technique for the rapid assessment of fibre quality in all forest species.

Another particular embodiment of the invention is the use of the invention on hybrid spruce specifically, although the invention is likely usable for all forest species.

One embodiment of the method invention described herein is related to DNA-based tests—for all wood quality parameters important for the desirability of wood for various solid wood and pulp and paper product applications—which enables acceleration of the process of assessment of natural populations and enables the early selection of elite lines for plantation establishment.

In another embodiment of the invention, the nucleic acid-based tests for difficult and expensive to measure traits (wood quality traits are an excellent example) provides a highly increased capability for cost reduction and product enhancement in selecting superior tree families.

Materials and Methods

Tree Sampling and Site Assessments Across the Introgression Zone

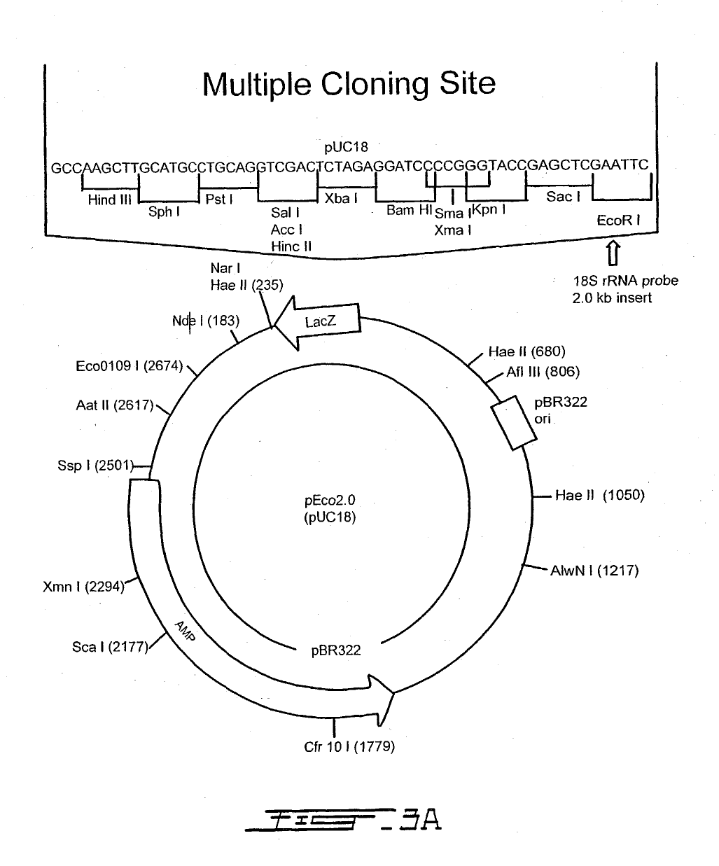

Seven site locations were identified west to east across the introgression zone based on desired spruce species (pure Sitka spruce, sitka and interior hybrids, and interior spruce—FIG. 2) and site quality utilizing Sutton et al.'s previous report as a guide (Sutton, B. C. S. et al., (1993) Can. J. For. Res. 24:278-285). Species were identified by looking at site location, gross morphology of the tree and by cone identification. The cones of each spruce species are different in the size and shape of both the cone and its scales (Farrar, J. L. Trees in Canada. Fitzhenry and Whiteside, Ltd. and Canadian Forest Service, pp. 95-107 (1997)). The tree's appearance (silhouette) and the cones of the hybrid species display characteristics of their parent species, but true species identification and degree of hybridization is difficult to determine by such gross methods. In terms of site quality, average or typical sites for the species were targeted.

Ecosystems, interacting complexes of living organisms and their physical/chemical environment, are grouped into biogeoclimatic zones, which are further divided into subzones (Steen, D. A. et al., (1997). A field guide to forest site identification and interpretation of the Cariboo forest region. B.C. Ministry of Forests, Victoria, B.C. Land management handbook No. 39). British Columbia is divided into 13 biogeoclimatic zones, which are large geographic areas with broadly homogeneous macroclimates (Forestry undergraduate society, Uni. Brit. Col. Forestry handbook for British Columbia, 4^{th} edition. D. W. Friesen and sons Ltd. pp 223-231 (1983)). Site quality was determined in the field by utilizing the biogeoclimatic ecosytem classification (BEC) method for site identification and analysis. This method utilizes vegetation, soils, climate, and topography in classifying ecosystems and estimating site quality [Banner, A. et al., (1993), A field guide to site identification and interpretation for the Prince Rupert forest region. B.C. Ministry of Forests, Victoria, B.C. Land management handbook No. 26). Site series was determined from estimated soil moisture and soil nutrient regimes. This was then related to site productivity (SIBEC) by estimating site index. Site index—a measure of the productivity of the stand—provides a standardized comparison of the productive potential between sites and is also determined through the site index curve method, which is a measure of tree height at fifty years breast height age (How to determine site index in silviculture. Participants work book, B.C. Ministry of forests, Forest renewal B.C. (1998)).

On average, 10 trees were sampled from each site, across a range of biogeoclimatic zones. The sampling consisted of collecting foliage samples, measuring tree heights and diameters at breast height (DBH), and removing 5 mm and 10 mm increment core samples at breast height.

Analysis of Fibre Properties

Spruce fibre properties were determined from the 10 mm increment core samples. At least one sample from each site was sectioned into age classes to monitor changes in fibre properties over time and to determine the juvenile/mature wood transition point for each of the species/hybrids studied. For all other samples only the 60-80 yr. age class was examined, as this was determined to be in the mature wood zone for each site. Fibres were released from the samples using a hydrogen peroxide/acetic acid maceration technique (Burkart, L. F. (1966) For. Prod. J. 16:52) and the resultant pulps were analyzed using an automated Fibre Quality Analyzer instrument to determine length weighted fibre lengths (LWFL) and fibre coarsenesses according to previously described protocols [Morrison, D. et al., (1998) Paprican PPR 1403).

Isolation of Genomic DNA and the Eco2.0 Probe

Genomic DNA from hybrids and pure Sitka and interior spruce was obtained using a FastPrep instrument (Bio 101 Inc.) according to the manufacturers standard protocols. The Eco2.0 rDNA probe-containing plasmid (pEco2.0—obtained courtesy of C. H. Newton, B.C. Research Inc., Vancouver) was isolated, purified and cloned into E. coli according to methods described in Sambrook et al. (Sambrook, J. et al., (1990) Molecular Cloning. A laboratory manual, 2^{nd} edition, Cold Spring Harbor Laboratory Press).

Restriction Digests

Both spruce genomic DNA samples and pEco2.0 were each digested with the appropriate restriction enzymes (HinD III and Eco R I respectively) according to the method of Hanish and McClelland (Hanish, J. et al., (1988) Gene Anal. Tech. 5:105). Approximately 25 ng of DNA was incubated at 37° C. in KGB buffer (1 M potassium glutamate, 250 mM Tris acetate pH 7.5, 100 mM magnesium acetate, 0.5 mg/ml BSA fraction 5, 5 mM β-mercaptoethanol) with 1 U of restriction enzyme for 2 hr and the reaction stopped with 0.5 M EDTA. Digests were analyzed on 1% agarose gels according to the method described in Sambrook et al., (1990).

Polymerase Chain Reaction

PCR was performed using M13 forward and reverse primers according to standard RAPD protocols.

Southern Hybridization

Southern blotting and Eco2.0 hybridization were performed according to Sutton et al., (1991) using the ECL detection system (Amersham).

Densitometry

Quantitation of bands following autoradiography was carried out using the AlphaInnotech AlphaImager 2000 documentation and analysis system. Using the “1-D multi” menu, integrated optical densities (OD)- were obtained, measured and scored for the total area of each band and its percentage value for all bands present in the lane.

DNA Sequencing

Terminal sequencing of the pEco2.0 plasmid was performed according to standard dideoxy-nucleotide termination protocols by the Nucleic Acid and Protein Services of the Biotechnology Laboratory at University of British Columbia. Since pEco2.0 is based on the pUC 18 vector, standard M13 forward and reverse primers were used for sequence priming.

Results and Discussion

Purification and Analysis of pEco2.0

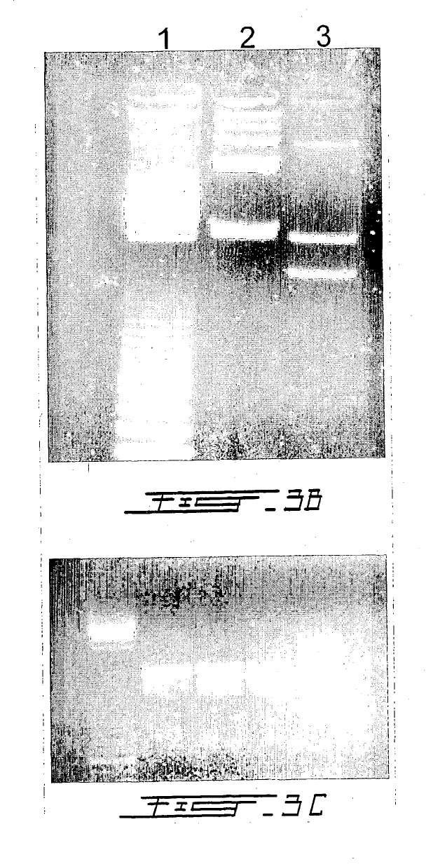

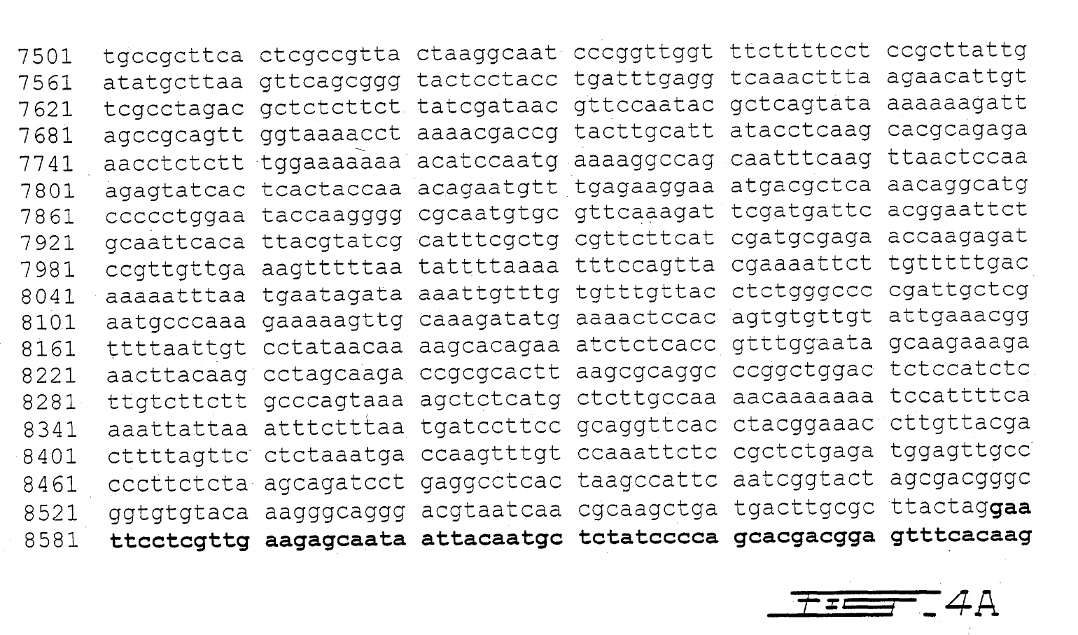

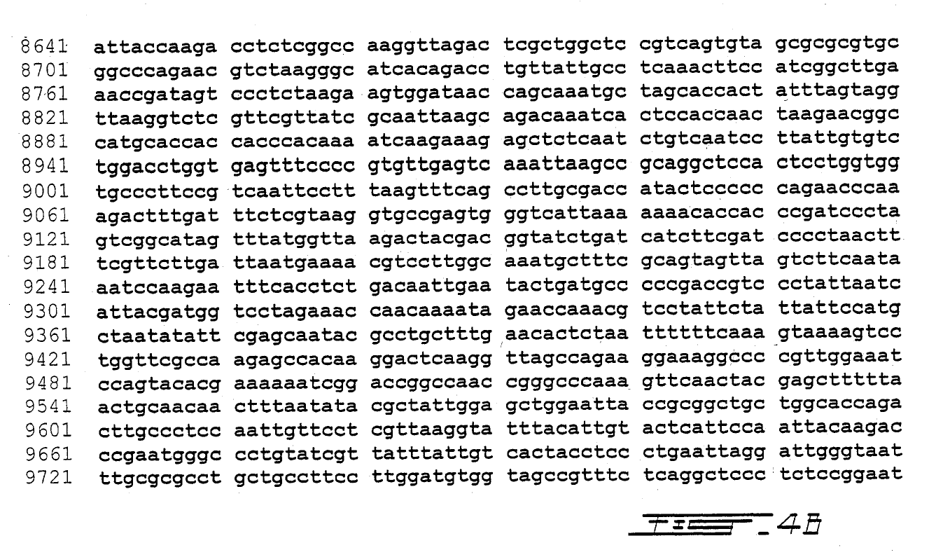

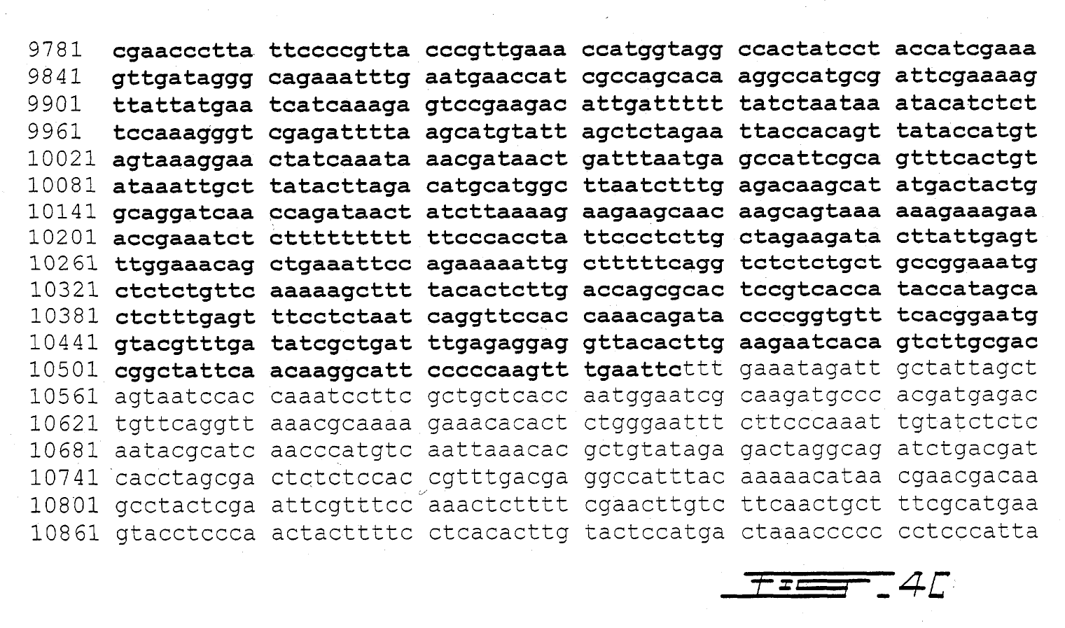

FIG. 3A shows a schematic of the pUC 18—based plasmid pEco2.0 and the insertion point of the probe into the multiple cloning site (MCS). In part B of FIG. 3, a 1% agarose gel analysis of the purified plasmid and its digestion products is presented. Lane 1: 100 bp markers (Pharmacia-Amersham); Lane 2: purified pEco2.0 (upper bands represent supercoiled versions of the plasmid); Lane 3: pEco2.0 digested to completion with restriction enzyme Eco R I to release the 2 kb yeast rDNA probe fragment. The analysis demonstrates that the. plasmid is sufficiently pure for probing and sequencing reactions. FIG. 3C shows a PCR amplification experiment using the pEco2.0 plasmid as template and M13 forward and reverse primers. FIG. 4 presents a fragment of Saccharomyces cerevisiae chromosome XII cosmid reading frame ORF YLR154c taken from genbank acc#Z73326. The complete sequence of the Eco2.0 probe is highlighted in bold face. FIG. 5 shows sequence analysis results for the pEco2.0 plasmid obtained using M13 forward and reverse primers.

Characterization of Sitka/Interior Spruce rDNA Polymorphisms

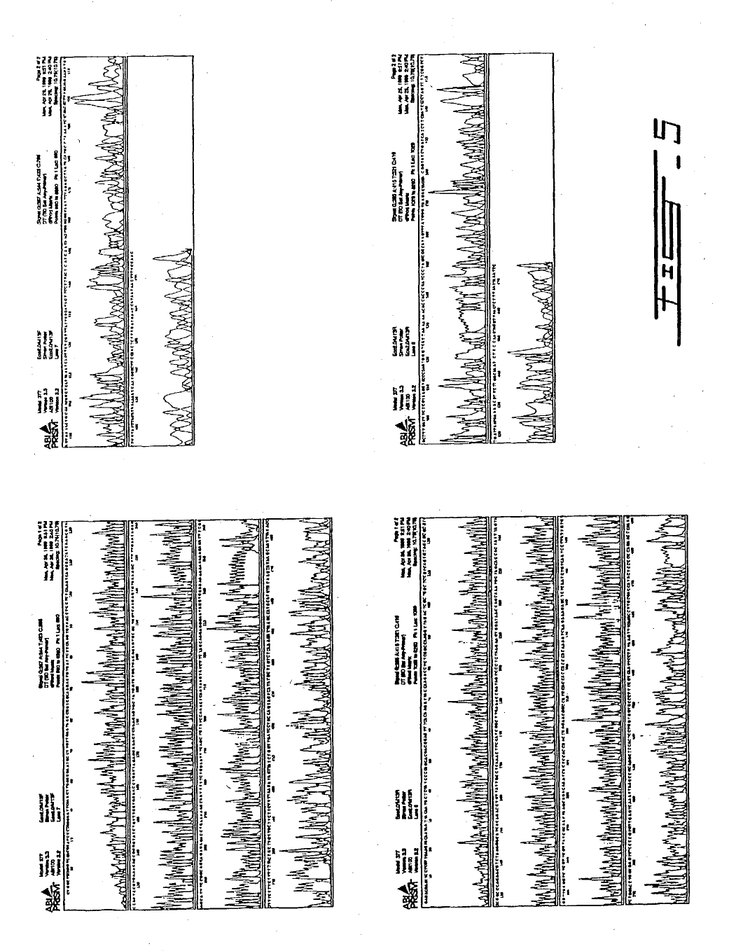

To confirm observations that the 2 kb yeast rDNA fragment could be used to distinguish Sitka and interior spruce species, Southern hybridizations of total DNA digested with HinD III were performed as previously described. The genomic DNA samples used were obtained from live tissue taken from the sampled trees using FastPrep instrument standard protocols. FIG. 6 shows a typical Southern hybridization obtained using the purified 2 kb rDNA probe and HinD III—digested genomic DNA samples taken from trees sampled across the zone. Lanes (L-R): Site 1/tree 4, 2/5*, 3/4, 4/9, 5/2, 6/1, 7/3. (*Tree 2/5 was not included in the analysis as repeated isolations failed to produce viable DNA).

Clearly, the pEco2.0 rDNA probe detects a polymorphic region of spruce DNA which is quite distinct in interior and Sitka species. The interior sample gives five bands of varying intensity after hybridization with the probe, whereas the Sitka sample gives only three. Bands 4 and 5, which are diagnostic of Sitka spruce, can be used in conjunction with chloroplast and mitochondrion—specific DNA markers to unambiguously differentiate between the species. Bands 1, 2 and 3 however are common to both species but are present in different relative intensities (FIG. 6). Densitometry of Band 2 was then used to estimate degrees of introgression exhibited by the hybrid trees sampled across the zone.

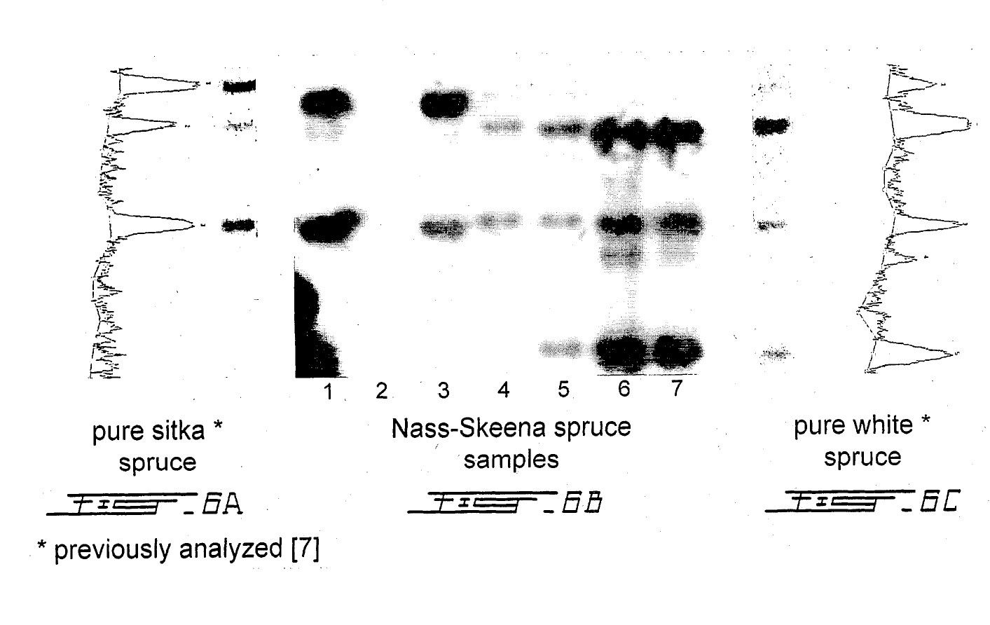

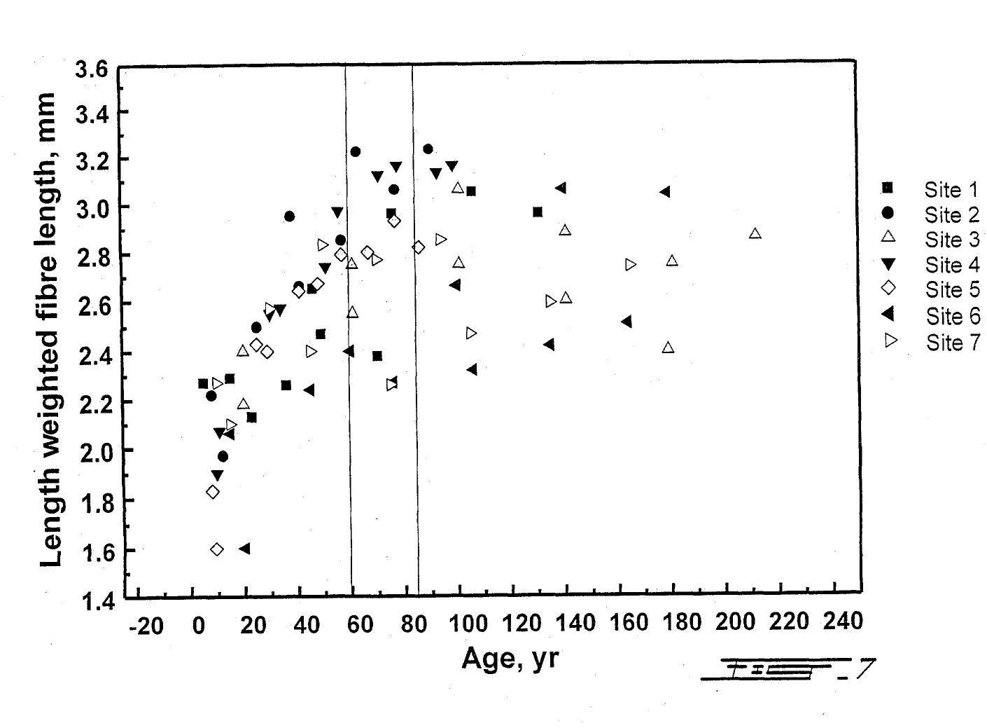

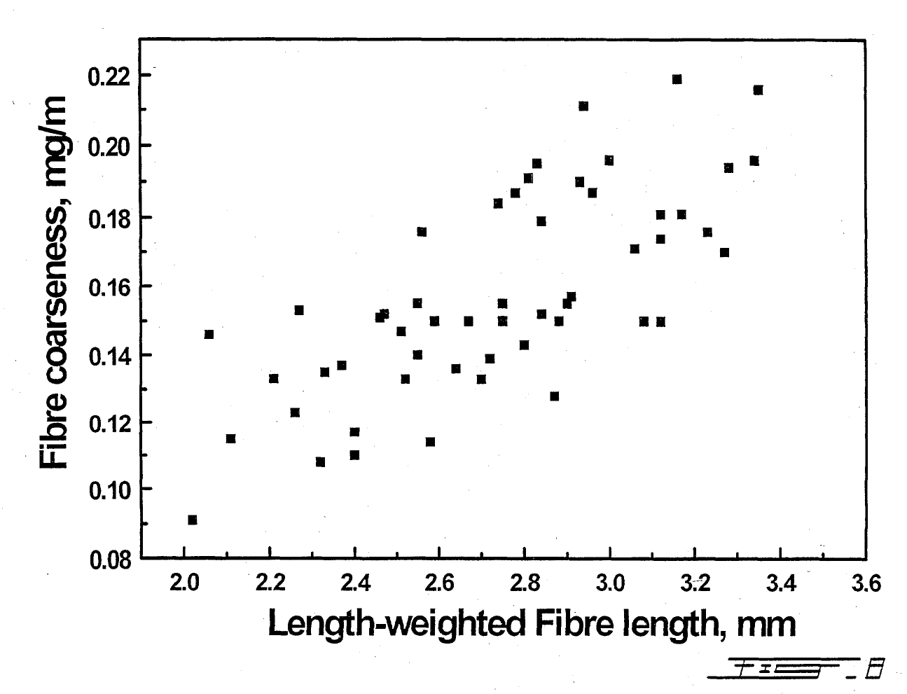

In order to compare the fibre properties of these hybrids, it is necessary to determine the juvenile:mature wood transition point by monitoring the change in fibre properties with age. This is shown in FIG. 7. Not surprisingly the juvenile:mature wood transition is somewhat site/hybrid dependent. However, by selecting age 60-80 yrs. (grey area in FIG. 7), it is possible to directly compare between these samples. Observed fibre lengths ranged from 3.34 mm in the west to 2.33 mm in the east (N.B.—these fibre length values were obtained from increment core samples and will, therefore, be shorter than actual whole log values). These length weighted fibre length values (60-80 years) obtained for all 54 trees examined in the study are plotted against the corresponding fibre coarseness values in FIG. 8. Fibre coarseness, a weight to length ratio, is an indirect measure of cell wall thickness and hence the important parameter of fibre collapsibility. Within a species, it is expected that a positive linear correlation should exist between length and coarseness, i.e. shorter fibres are finer whereas larger fibres should be coarser. FIG. 8 demonstrates that this holds true for the introgression zone population, with a strong positive correlation observed (Pearson coefficient 0.74, p=0.000).

The fibre parameters for the specific trees chosen for genetic hybridization analysis via DNA-probing are presented in Table I.

TABLE I
Fibre properties, band intensities and site index values for the
sampled spruce hybrids

| | | | Fibre | Fibre | Band | | | | |
|---|---|---|---|---|---|---|---|---|---|
| | Tree | Site | Length | Coarseness | Intensity | | | | |
| Site | # | Index | (mm) | (mg/m) | (U) Band 1 | Band2 | Band3 | Band4 | Band5 |
| 1 | 4 | 29 | 2.37 | 0.137 | 3390 | 843 | 2233 | — | 596 |
| 3 | 4 | 41 | 2.90 | 0.155 | 3287 | 826 | 2492 | — | 169 |
| 4 | 9 | 16.5 | 2.81 | 0.191 | 1362 | 2572 | 2509 | 715 | 1146 |
| 5 | 2 | 20.5 | nd* | nd* | — | — | — | — | |
| 6 | 1 | 15.2 | 2.55 | 0.140 | 430 | 3531 | 2324 | 940 | 2294 |
| 7 | 3 | 25.3 | 2.33 | 0.135 | — | 4237 | 2218 | 1022 | 2549 |

*Tree not suitable for analysis as it is too young (55 yr).

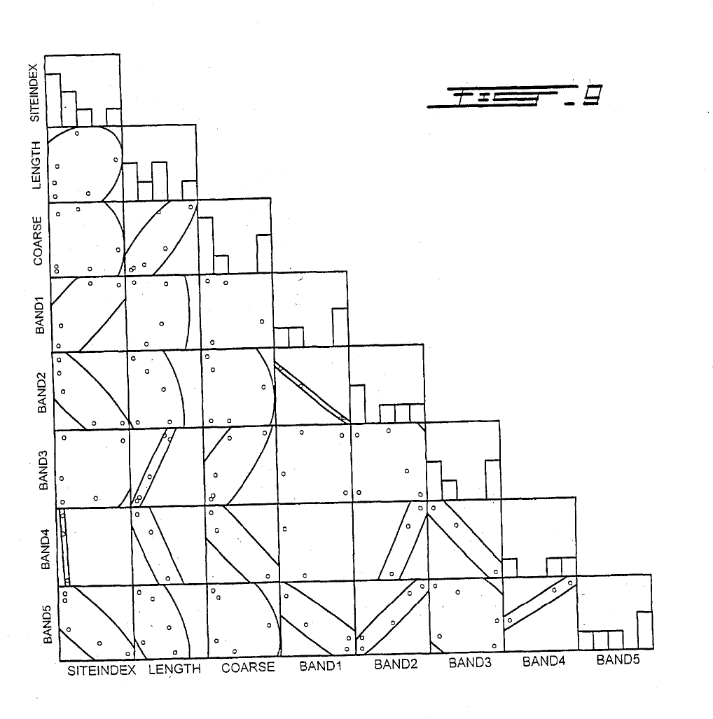

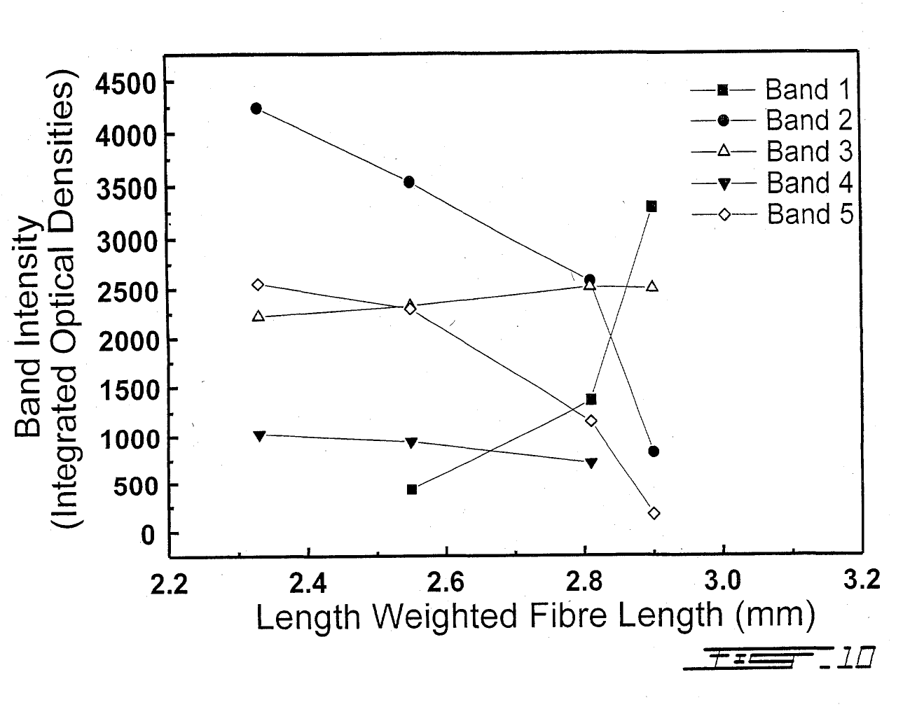

These data were then compared with the band intensities obtained from the Southern blot analysis and the resultant plots are all shown in FIG. 9 (Table II presents Pearson correlation matrix for FIG. 9). FIG. 10 shows one such plot—the relationship between fibre length and band intensity. It can be seen that in each case, there is a linear relationship between the estimated level of genetic introgression (from the DNA-probing) and the measured fibre properties of the selected trees (example correlation coefficients: for fibre length to Band 2 intensity (excluding site 1 outlier)=−0.87G; for fibre coarseness to Band 3 intensity=0.843).

TABLE II
Pearson correlation matrix for FIG. 9 including site 1
outlier point (excluding site 1)

| | Site | | | | | | | |
|---|---|---|---|---|---|---|---|---|
| | Index | Length | Coarse | Band 1 | Band 2 | Band 3 | Band 4 | Band5 |
| Site | 1.000 | | | | | | | |
| Index | | | | | | | | |
| Length | 0.308 | 1.000 | | | | | | |
| Coarse | −0.009 | 0.859 | 1.000 | | | | | |
| Band 1 | 0.881 | −0.015 | −0.228 | 1.000 | | | | |
| Band 2 | −0.874 | −0.398 | −0.134 | −0.999 | 1.000 | | | |
| | | (−0.876) | | | | | | |
| Band 3 | 0.320 | 0.985 | 0.843 | −0.101 | −0.333 | 1.000 | | |
| Band 4 | −0.946 | −0.977 | −0.984 | −1.000 | 0.985 | −0.994 | 1.000 | |
| Band 5 | −0.863 | −0.558 | −0.328 | −0.937 | 0.973 | −0.510 | 0.996 | 1.000 |

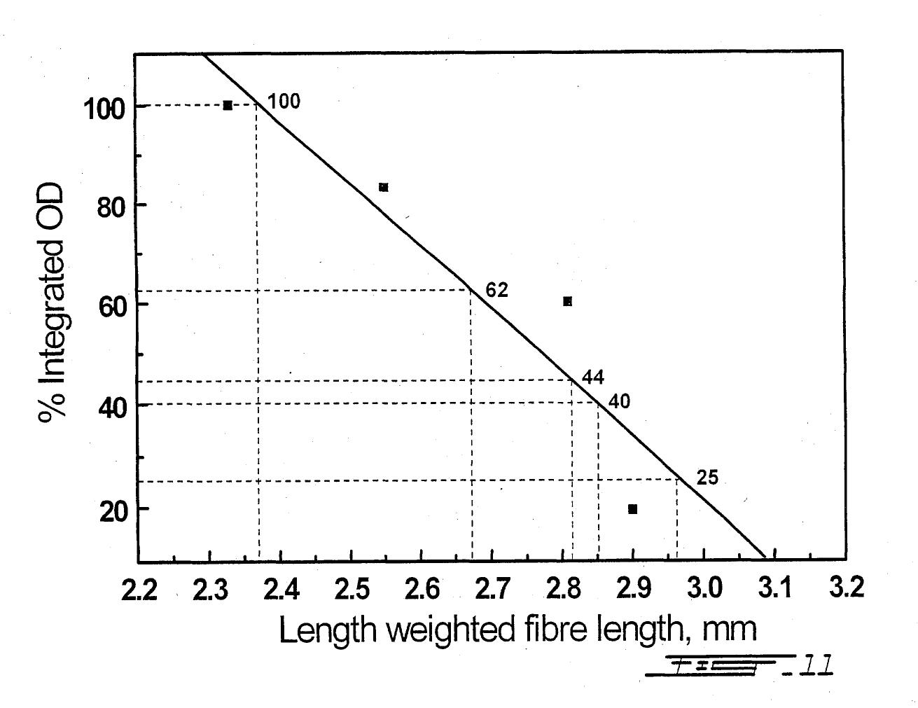

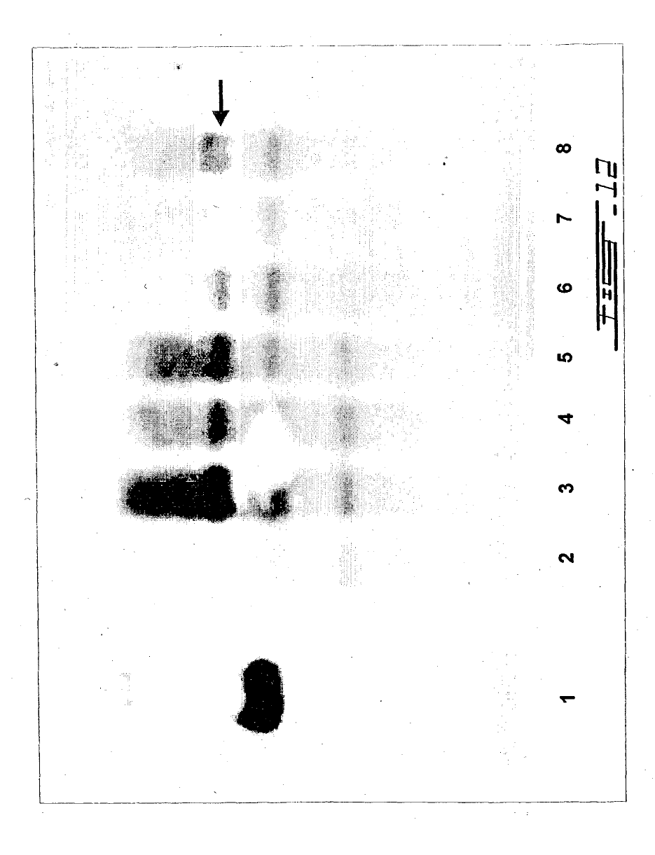

The data obtained from the first blot shown in FIG. 6 were used to develop -a standard curve relationship between relative band intensity (integrated optical densities as a relative percentage of total signal in the lane) and length-weighted fibre length across the introgression zone, FIG. 11. A second blotting experiment was then performed using different samples from six of the seven sites and relative % band intensities were obtained (FIG. 12). The intensity of Band 2 was chosen—as this was the clearest band on the second blot and showed a good positive correlation with fibre length on the first blot—and the intensity values obtained for each spruce sample were interpolated onto the standard curve to predict the length-weighted fibre length values for the corresponding trees. Predicted values for fibre length were then compared to actual fibre length values obtained from FQA analysis on cores obtained from the sampled trees and the results of the comparison are presented in Table III. With one exception, site 4 tree 5, it can be clearly seen that the DNA-based predictive analysis is reproducibly precise for this particular trait.

TABLE III
Prediction of hybrid spruce fibre length using DNA
probe band intensity

| | Integrated | Predicted | Actual | |
|---|---|---|---|---|
| | o.d., % | fibre length | fibre length | Difference |
| Tree# | (Band 2) | mm (Blot) | mm (FQA) | mm |
| 2-4 | 25 | 2.96 | 2.94 | +0.02 |
| 4-5 | 40 | 2.85 | 3.16 | −0.31 |
| 5-8 | 44 | 2.81 | 2.80 | +0.01 |
| 6-4 | 62 | 2.67 | 2.64 | +0.03 |
| 7-9 | 100 | 2.38 | 2.47 | −0.09 |

A linear regression was produced using the Band 2 intensity data from Blot 1. Blot 2 Band 2 intensity data were then interpolated using the linear regression and corresponding predicted fiber lengths noted. Actual fibre lengths were determined from macerated cores as described in the text.

Further study must now be employed to examine whether this phenomenon is specific to the particular hybrids within the Nass-Skeena zone or if it will prove to be generally applicable to any species hybrids providing that a significant difference in the trait of interest exists between the two pure species which comprise that hybrid. However, it is worthy of note that, on the second blot (FIG. 12), Lane 2 contains genomic DNA isolated from a tree identified morphologically as a Black spruce sample obtained from a site at Nass-Skeena. This DNA, when tested with the pEco2.0 probe, gives a pattern of bands identical to that seen for interior spruce samples from B.C., suggesting that the probe may potentially also be able to distinguish hybrids of other spruce species.

Previous work on interior B.C. species such as subalpine fir has shown a positive and statistically significant correlation between site quality, as measured by site index, and fibre properties in the 65-85 year age class (Watson, P. A., et al., (October 1999) Paprican PPR 1454). In this study, the biogeoclimatic subzone, and hence site quality, variations were significant as shown in Table IV.

TABLE IV
Site quality data

| | | | Site | Sibec | |
|---|---|---|---|---|---|
| Site | Biogeoclimatic | Spruce | Quality | Site | Measured |
| # | Zone | Species* | Estimates | Index | Site Index |
| 1 | CWHvm1 | Sitka | good | 28 | 29 |
| 2 | ICHmc1 | Ss × Sw | good | 24 | 24.8 |
| 3 | CWHws1 | Ss × Sw | good | 32 | 41 |
| 4 | ICHmc2 | Ss × Sw | good | 21 | 16.5 |
| 5 | ICHmc2 | Ss × Sw | medium | 21 | 20.5 |
| 6 | SBSmc2 | Sw × Se | medium | 18 | 15.2 |
| 7 | SBSmc2 | Sw × Se | medium | 18 | 15.3 |

*Spruce species according to the field guidebooks.

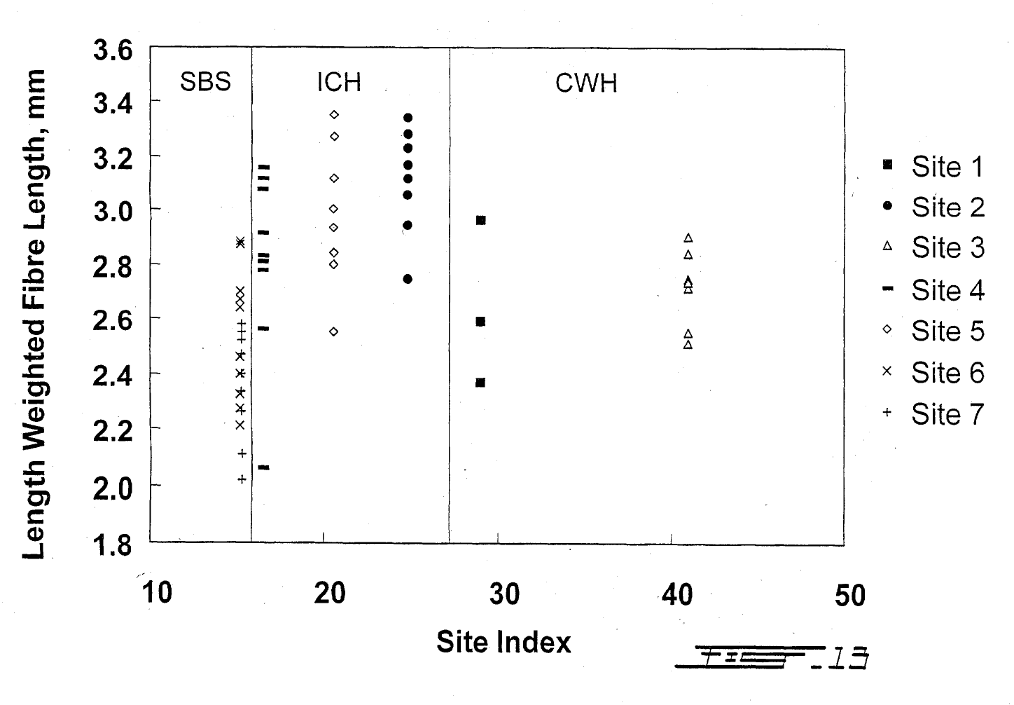

Fibre length (60-80 yr) is plotted as a function of site index in FIG. 13. It is evident that there is no correlation between site productivity and fibre length across biogeoclimatic zones, indicating that the genetic differences between these hybrids mask any significant macroenvironmental effect.

The results obtained in this study confirm the utility of nuclear DNA based probes as marker systems to monitor the extent of genetic hybridization between certain forest species of commercial interest. More importantly, however, they additionally suggest that such probes may be useful also as markers for industrially relevant wood quality parameters within species in both assessment and predictive modes. If found to be generally applicable across many species, DNA marker systems such as the one described herein could become highly valuable tools for the rapid assessment of natural populations and for the screening of cuttings, somatic embryos etc. for wood quality potential prior to implementation in plantation programs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for identifying tree lineage capable of expressing desired biological and/or biochemical phenotypes comprising the steps of:
a) obtaining a nucleic acid sample from trees of pure species and/or hybrid thereof;
b) obtaining a restriction pattern of restriction fragments by subjecting said nucleic acid sample of step a) to at least one restriction enzyme, wherein said restriction enzyme maximizes differences between said restriction pattern of pure species and/or hybrid thereof;
c) visualizing said restriction pattern of step b) by submitting the treated nucleic acid sample of step b) to at least one labeled probe for complementary hybridization between said probe and said nucleic acid sample, wherein said probe allows for detection of the degree of hybridization and/or different intensity of said restriction fragments between trees of pure species and/or hybrid thereof; and
d) correlating said restriction pattern and/or intensity of restriction fragments of step c) to at least one selected biological and biochemical phenotype of said tree, wherein said phenotype is associated with a genetic locus correlated with said phenotype.

2. The method according to claim 1, wherein said correlating of step d) further comprises a standard curve for predictive relationship between said restriction pattern and/or intensity of restriction fragments, and said phenotype.

3. The method according to claim 1, wherein said nucleic acid sample is selected from the group consisting of DNA and RNA.

4. The method according to claim 1, wherein said tree of pure species and/or hybrid thereof is naturally or artificially produced.

5. The method according to claim 1, wherein said nucleic acid sample of step a) is obtained from a leaf, cambium, root, bud, stem, cork, phloem or xylem.

6. The method according to claim 1, wherein said tree is of the genus Picea.

7. The method according to claim 1, wherein- said tree is of the genus Populus, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja, or Canya.

8. The method according to claim 1, wherein said step c) further comprises measurement of intensity of said restriction fragments.

9. The method according to claim 1, wherein said biological or biochemical phenotype is selected from the group consisting of fiber length, wood density, fiber collapsibility, fiber coarseness, cell wall thickness, growth rate, lignin content, guaiacyl lignin content, syringyl lignin content, carbohydrate content, kraft pulp yield, mechanical pulp energy demand, chemical uptake for chemical pulping, extractive content, and extractive compounds.

10. The method according to claim 1, wherein said probe is Eco2.0.

11. Use of a pattern of restriction fragments obtained by subjecting a nucleic acid sample from trees of pure species and/or hybrid thereof to at least one restriction enzyme for identifying tree lineage capable of expressing desired biological and/or biochemical phenotypes, wherein said restriction enzyme maximizes differences between said pattern of restriction fragments from pure species and/or hybrid thereof.

12. The use of claim 11, wherein said nucleic acid sample is selected from the group consisting of DNA and RNA.

13. The use according to claim 11, wherein said tree of pure species and/or hybrid thereof is naturally or artificially produced.

14. The use according to claim 11, wherein said nucleic acid sample of step a) is obtained from a leaf, cambium, root, bud, stem, cork, phloem or xylem.

15. The use according. to claim 11, wherein said tree is of the genus Picea.

16. The use according to claim 11, wherein said tree is of the genus Populus, Betula, Abies, Larix, Taxus, Ulmus, Prunus, Quercus, Malus, Arbu tus, Salix, Platanus, Acer, Tsuga, Pseudotsuga, Pinus, Fraxinus, Eucalyptus, Acacia, Abrus, Cupressus, Fagus, Juniperus, Thuja, or Canya.

17. The use according to claim 11, wherein said biological or biochemical phenotype is selected from the group consisting of fiber length, wood density, fiber collapsibility, fiber coarseness, cell wall thickness, growth rate, lignin content, guaiacyl lignin content, syringyl lignin content, carbohydrate content, kraft pulp yield, mechanical pulp energy demand, chemical uptake for chemical pulping, extractive content, and extractive -compounds.

18. Use of a Eco2.0 probe as a marker for predicting wood or fiber quality of tree samples of pure species and/or hybrid thereof.

19. The use of claim 18, wherein said tree of pure species and/or hybrid thereof is naturally or artificially produced.

20. The use of claim 18, wherein said tree is of the genus Picea.

21. The use of claim 18, whereinin said tree is of the genus Populus, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja, or Canya.

22. The use according to claim 18, wherein said wood or fiber quality is selected from the group consisting of fiber length, wood density, fiber collapsibility, fiber coarseness, cell wall thickness, growth rate, lignin content, guaiacyl lignin content, syringyl lignin content, carbohydrate content, kraft pulp yield, mechanical pulp energy demand, chemical uptake for chemical pulping, extractive content, and extractive compounds.

23. A method of screening a plurality of trees of diverse phenotypes which comprises the steps of:
a) Characterizing wood quality of at least two trees with different degree of hybridization;
b) Developing a standard curve from said trees for a predictive relationship between restriction patterns and phenotypes;
c) assessing a plurality of natural species hybrids for restriction patterns of a plurality of hybridization markers;
d) comparing said restriction patterns of said hybrids with said standard curve to deduce a phenotype of said hybrids; and
e) harvesting said hybrids based on predicted phenotypes.

24. A method of producing a plurality of clonal trees having predictable, consistent and/or enhanced wood or fibre quality properties, which comprises the steps of:
a) characterizing wood quality of at least two trees with different degree of, hybridization;
b) developing a standard curve from said trees for a predictive relationship between restriction patterns and phenotypes;
c) assessing a plurality of natural species hybrids for restriction patterns of a plurality of hybridization markers;
d) comparing said restriction patterns of- said hybrids with said standard curve to deduce a phenotype of said hybrids;
e) obtaining a plurality of progeny trees from said parental trees by performing cross-pollination or somatic embryogenesis; and
f) propagating somatic embryos of said progeny trees obtained in step e) to produce a plurality of clonal trees, essentially all of said clonal trees having predictable, consistent and/or enhanced wood or fibre quality properties.

25. The method according to claim 23, wherein said parent tree is naturally or artificially produced.

26. The method according to claim 23, wherein said parent tree is of the genus Picea.

27. The method according to claim 23, wherein said tree is of the genus Populus, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja, or Canya.

28. A stand of clonal trees with enhanced wood or fibre properties produced by the method of claim 23, 24, 25, 26 or 27, the genome of said trees containing a restriction pattern, said restriction pattern being the same restriction pattern associated with said enhanced wood or fibre properties.
